(19) United States
(12) Patent Application Publication
(10) Pub. No.: US 2025/0348756 A1
(43) Pub. Date: Nov. 13, 2025

(54) EXPLANATION-ASSISTED DATA AUGMENTATION FOR GRAPH NEURAL NETWORK TRAINING

(71) Applicant: NEC Laboratories America, Inc., Princeton, NJ (US)

(72) Inventors: Wei Cheng, Princeton Junction, NJ (US); Haifeng Chen, West Windsor, NJ (US); Dongsheng Luo, Miami, FL (US)

(21) Appl. No.: 19/199,864

(22) Filed: May 6, 2025

Related U.S. Application Data
(60) Provisional application No. 63/646,137, filed on May 13, 2024.
(60) Provisional application No. 63/649,572, filed on May 20, 2024.

(51) Int. Cl. G06N 3/0985; B60W 60/00; G16H 50/20

(52) U.S. Cl. CPC G06N 3/0985; B60W 60/0011; G16H 50/20

(57) ABSTRACT

Systems and methods for explanation-assisted data augmentation for training graph neural networks. The GNN can be trained using a training dataset to generate explainer subgraphs of labeled graphs. The explainer subgraphs can be transformed into perturbed subgraphs by utilizing explanation assisted empirical risk minimization (EA-ERM) learned by the GNN to generate an augmented training dataset. The GNN can be further trained with the augmented training dataset and the training dataset to perform downstream tasks using input data with corresponding labels.

### RELATED APPLICATION INFORMATION

This application claims priority to U.S. Provisional App. No. 63/646,137, filed on May 13, 2024, and U.S. Provisional App. No. 63/649,572, filed on May 20, 2024; incorporated herein by reference in their entirety.

### BACKGROUND

### Technical Field

The present invention relates to training artificial intelligence models and more particularly to explanation-assisted data augmentation for graph neural network training.

### Description of the Related Art

Graphs can represent relationships between entities in a wide range of applications including social networks, biology, and finance. To effectively leverage the rich relational information encoded in graphs, various graph neural network (GNN) architectures have been developed. However, optimization of the training procedures and the training data for GNNs is still a developing field in machine learning and artificial intelligence.

### SUMMARY

According to an aspect of the present invention, a computer-implemented method is provided for training a graph neural network (GNN), having, training the GNN using a training dataset to generate explainer subgraphs of labeled graphs, transforming the explainer subgraphs into perturbed subgraphs by utilizing explanation assisted empirical risk minimization (EA-ERM) learned by the GNN to generate an augmented training dataset, and training the GNN with the augmented training dataset and the training dataset to perform downstream tasks using input data with corresponding labels.

According to another aspect of the present invention, a system for training a graph neural network (GNN), including, a memory device, one or more processor devices operatively coupled with the memory device to perform operations having, training the graph neural network (GNN) using a training dataset to generate explainer subgraphs of labeled graphs, transforming the explainer subgraphs into perturbed subgraphs by utilizing explanation assisted empirical risk minimization (EA-ERM) learned by the GNN to generate an augmented training dataset, and training the GNN with the augmented training dataset and the training dataset to perform downstream tasks using input data with corresponding labels.

According to yet another aspect of the present invention, a non-transitory computer program product is provided including a computer-readable storage medium including a program code for training a graphical neural network (GNN), wherein the program code when executed on a computer causes the computer to perform operations having, training the GNN using a training dataset to generate explainer subgraphs of labeled graphs, transforming the explainer subgraphs into perturbed subgraphs by utilizing explanation assisted empirical risk minimization (EA-ERM) learned by the GNN to generate an augmented training dataset, and training the GNN with the augmented training dataset and the training dataset to perform downstream tasks using input data with corresponding labels.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram showing a high-level overview of a method for explanation-assisted data augmentation for graph neural network training, in accordance with one embodiment of the present invention;

FIG. 2 is a block diagram showing the overall algorithm for training the GNN for explanation-assisted data augmentation for graph neural network training, in accordance with an embodiment of the present invention; and

FIG. 3 is a block diagram showing a system implementing practical applications for explanation-assisted data augmentation for graph neural network training, in accordance with an embodiment of the present invention;

FIG. 4 is a block diagram showing software and hardware components of a computer system for explanation-assisted data augmentation for graph neural network training, in accordance with an embodiment of the present invention;

FIG. 5 is a block diagram showing a computer system for explanation-assisted data augmentation for graph neural network training, in accordance with an embodiment of the present invention; and

FIG. 6 is a block diagram showing a structure of deep neural networks for explanation-assisted data augmentation for graph neural network training, in accordance with an embodiment of the present invention.

### DETAILED DESCRIPTION OF PREFERRED EMBODIMENTS

In accordance with embodiments of the present invention, systems and methods are provided for explanation-assisted data augmentation for graph neural network (GNN) training.

In an embodiment, the GNN can be trained using a training dataset to generate explainer subgraphs of labeled graphs. The explainer subgraphs can be transformed into perturbed subgraphs by utilizing explanation assisted empirical risk minimization (EA-ERM) learned by the GNN to generate an augmented training dataset. The GNN can be further trained with the augmented training dataset and the training dataset to perform downstream tasks using input data with corresponding labels.

Graphs are used to represent relationships between entities in a wide range of applications including social networks, biology, and finance. To effectively leverage the relational information encoded in graphs, various graph neural network (GNN) architectures have been developed, such as methods based on convolutional neural networks, recurrent neural networks, and transformers. To enhance the generalization capabilities and avoid overfitting during training of GNNs, developing data augmentation (DA) techniques are in progress such as methods involving subgraph explanations.

DA can enlarge the training set through label-preserving transformations. These techniques enhance generalization, especially when the size of the original training set is limited. A fundamental idea in DA is that labels are invariant to domain-specific transformations. For instance, in many image classification tasks, it is expected that the output label remains invariant to specific affine transformations of the original image, such as rotation and scaling.

DA can be used in graphs. A large class of graph augmentation methods can be categorized as rule-based, learning-based methods, and explanation-assisted data augmentation (EA) methods. Rule-based methods can randomly drop or crop a subset of features in the original graph, and substitute the graphs. Learning-based methods can use GNNs to learn edge importance. For example, local augmentation can learn the conditional distribution of the node under its neighbors. EA-DA methods construct label-preserving transformations based on explanations. For instance, given the ground-truth explanation, a generative adversarial network (GAN) can be used to generate image augmentations conditioned on the explanation sub-image. Other EA-DA methods (also called explanation-guided DA) can generate explanation-assisted augmentations. However, such methods do not reflect the importance of each token in natural language processing, and fail to preserve structural and semantic properties of the graph.

DA techniques can be used in non-graphical domains for learning over graphs. However, in contrast to DA in non-graphical domains, slight edge perturbations in graphs often lead to out-of-distribution samples. For instance, in molecular structures modeled as graphs, any edge perturbation that connects a carbon atom to more than four other atoms yields an out-of-distribution sample. Furthermore, classification labels are highly sensitive to edge modifications, and a single edge removal or addition may significantly change the properties of the molecular structure. Thus, it is challenging to identify label-preserving transformations which preserve structural and semantic properties in learning over graphs that are in-distribution augmentations due to the complexity of graphs.

Moreover, learning over non-graphical domains with out-of-distribution augmentations can lead to increased sample complexity and lower efficiency. In machine learning, sample complexity can measure the number of training examples a learning algorithm uses to achieve a certain level of accuracy or performance. A lower sample complexity means fewer training examples which can lead to increased computational cost efficiency in training.

Other methods can use gradients to extract explanations but can be limited to a model. To overcome this, model-agnostic methods, including perturbation-based methods, can be utilized. Perturbation-based methods, generate perturbations to determine which features and subgraph structures are important. Perturbation invariance is a goal for learning with perturbation-based methods, which can refer to a model's robustness to malicious inputs crafted to mislead the model. The notion of perturbation invariance is analogous to transformation invariances, such as rotation and scaling invariances, observed in image classification. Empirical risk minimization (ERM) is a learning paradigm in machine learning utilized to determine an optimal model that minimizes the average error or loss on a given training set, and treating this average as an estimate of the model's overall risk.

The present embodiments address the issues with other augmentation methods for graph learning. The present embodiments propose explanation-assisted data augmentation (EA-DA), specifically, explanation-assisted empirical risk minimization (EA-ERM) that can achieve perturbation invariance with less sample complexity for learning over graph structured inputs. The present embodiments introduce DA techniques that leverage the notion of subgraph explainability to enlarge the training set via label-preserving graph perturbations. By training the GNN with the augmented training dataset, the data efficiency and accuracy of the GNNs training and the GNNs' learning of target datasets are increased when compared to other augmentation techniques (e.g., edge inserting, edge dropping, node dropping, feature dropping, mixup, etc.). In contrast, the performance of the GNN worsens when the GNN is trained with out of distribution augmentations which can result by performing other augmentation techniques through random edge addition, removal, etc.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

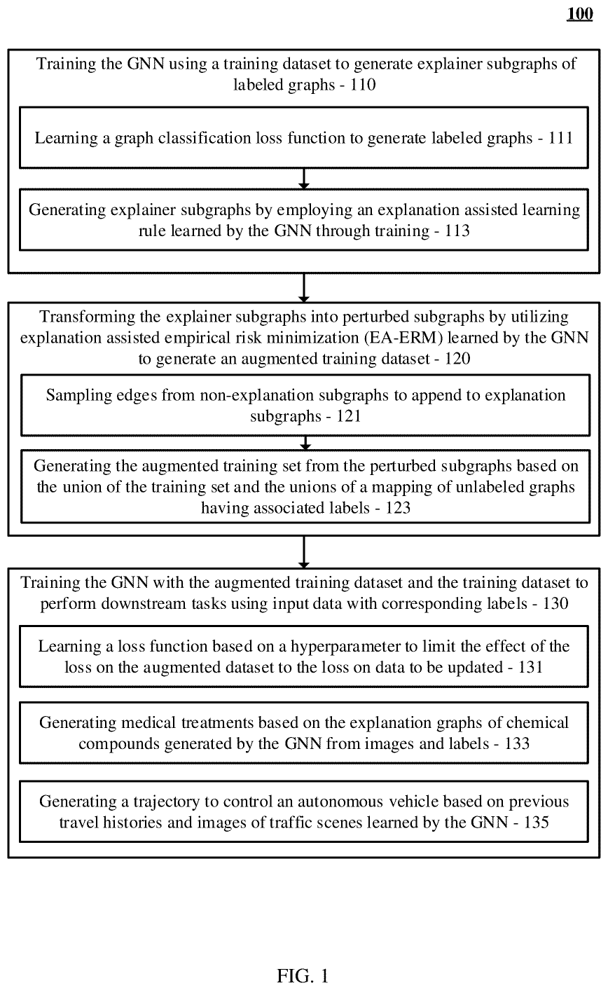

Referring now in detail to the figures in which like numerals represent the same or similar elements and initially to FIG. 1, a block diagram showing a high-level overview of a method for explanation-assisted data augmentation for graph neural network training, in accordance with one embodiment of the present invention.

In an embodiment, the GNN can be trained using a training dataset to generate explainer subgraphs of labeled graphs. The explainer subgraphs can be transformed into perturbed subgraphs by utilizing explanation assisted empirical risk minimization (EA-ERM) learned by the GNN to generate an augmented training dataset. The GNN can be further trained with the augmented training dataset and the training dataset to perform downstream tasks using input data with corresponding labels.

In block 110, the GNN can be trained using a training dataset to generate explainer subgraphs of labeled graphs.

To train the GNN using the training dataset to generate explainer subgraphs of labeled graphs, components of the training method can be defined.

A graph G is parametrized by a vertex set V={v₁, v₂, . . . , vₙ}, where n∈N, an edge set ε⊆V×V, a feature matrix X∈ [symbol] ^{n×d}, where the ith row Xi is associated with vᵢ and d is the feature dimension, an adjacency matrix Aε{0, 1}^{n×n}, where A_{i,j}= [symbol] ((vᵢ, vⱼ)∈ε), and a label Y∈γ, where γ is a finite set. The graph parameters (Y, A, X) and label Y are generated based on the joint distribution P_{Y,A,X}. The notation P_{Y,A,X} and P_{G} are used interchangeably. For a labeled graph G=(V, ε; Y, A, X), the corresponding graph without labels is denoted as G̅=(V, ε; Y, A, X). The induced marginal distribution of G̅ is P_{G̅} and its support is [symbol] .

A classification scenario can be characterized by P_{G}. A graph classifier for a classification scenario P_{G} is a function ƒ: [symbol] →γ, where G is the support of P_{G}. Given ϵ∈[0, 1], the classifier is called ϵ-accurate if P_{G}(ƒ(G̅)≠Y)≤ϵ.

A training set T is a collection of labeled graphs. The elements of the training set are generated independently and according to P_{G}. In an embodiment, the training dataset can be generated by a GNN trained with a learning rule for classification to convert input data (e.g., images, text, video, etc.) and labels into labeled graphs.

The labeled graphs can include semantic information about the underlying data converted into a graph. For example, for a graph converted from a chemical compound X, the nodes of the graph can represent different elements or compounds of the chemical compound X, while edges can represent the bonds between the elements or compounds. The overall label for such a graph can describe the chemical compound, X.

A learning rule is a procedure that takes the training set T as input, and outputs a graph classifier f(·) belonging to an underlying hypothesis class H. A generic learning rule can be L= [symbol] consists of a family of mappings Lₜ: Tₜ→ƒ(·), where input Tₜ={(G̅ᵢ, Yᵢ), i∈|t|} is called the training set, and the output ƒ: [symbol] →γ is a graph classifier belonging to the hypothesis class H.

The empirical risk minimization (ERM) is a subclass of the generic learning rule which can be defined as L_{ERM}= [symbol] ; where

$${L}_{ERM , t} ( {T}_{t} ) {=}^{Δ} arg {min}_{f ( . ) ∈ {H}^{t}} \frac{1}{t} {\sum }_{i = 1}^{t} 𝟙 ( f ( {\overline{G}}_{i} ) \neq {Y}_{i} ) , t ∈ ℕ .$$

In block 111, training the GNN using the training dataset by learning a graph classification loss function to generate labeled graphs.

In an embodiment, the GNN can be trained using a graph classification loss function such as cross entropy loss, soft-max function, etc. The graph classification loss trains the classification function of the GNN to minimize the error of the output Y of the GNN for corresponding unlabeled graph G̅. The GNN can implement the following frameworks: Graph Convolutional Network, Graph Isomorphism Network, Principal Neighbourhood Aggregation, and GraphSage. Other frameworks can be implemented.

In another embodiment, the training dataset can include the labeled graphs which can be obtained from pre-made training datasets (e.g., MU-TAG, Benzene, Fluoride, Alkane, D&D, PROTEINS, etc.).

In block 113, explainer subgraphs can be generated by employing an explanation assisted learning rule can be learned by the GNN through training.

To train a GNN to generate explainer subgraphs, the generation of explainer subgraphs using explanation-assisted ERM can be learned and defined with the following. Given a hypothesis class H, training set T, and explanation function Ψ(·), the EA-ERM learning rule produces L_{EA-ERM}(T) [symbol] (·), where:

$$\tilde{f} ( G ) {=}^{Δ} \{ \begin{matrix}{Y}_{exp} , {∃}_{i} ∈ [ t ] : Ψ ( {G}_{i} ) ⊆ G \\ f ( G ) , Otherwise\end{matrix} , f ( \cdot ) {=}^{Δ} {L}_{ERM} ( T ) , \qquad ( 2 )$$

where Yₑₓₚ is chosen randomly and uniformly from the set {Yᵢ|Ψ(Gᵢ)⊆G, i∈[T]}, and L_{ERM} denotes the (explanation-agnostic) ERM learning rule.

An explanation function is a mapping Ψ: G̅→2^{V}×2^{ε}, such that Ψ(G̅)=(Vₑₓₚ, εₑₓₚ), G̅∈ [symbol] , where Vₑₓₚ⊆V, εₑₓₚ=(Vₑₓₚ×Vₑₓₚ)∩ ε, and V and ε are the vertex set and edge set of G̅ respectively. For a given pair of parameters κ∈[0,1] and s∈ [symbol] , the explainer Ψ(·) is a (s, κ)-explainer if: I(Y; G̅| [symbol] _{Ψ(G̅)}) and E_{G}(|εₑₓₚ|)≤S where:

$$I ( Y ; \overline{G} \vert {𝟙}_{Ψ ( \overline{G} )} ) {=}^{Δ} {\sum }_{{g}_{exp}} {P}_{Ψ ( \overline{G} )} ( {g}_{exp} ) {\sum }_{y , \overline{g}} {P}_{Y , \overline{G}} ( y , \overline{g} \vert {g}_{exp} ⊆ \overline{G} ) \times log \frac{{P}_{Y , \overline{G}} ( y , \overline{g} \vert {g}_{e x p} ⊆ \overline{G} )}{{P}_{Y} ( y , \overline{g} \vert {g}_{e x p} ⊆ \overline{G} ) {P}_{\overline{G}} ( y , \overline{g} \vert {g}_{e x p} ⊆ \overline{G} )} .$$

To keep the analysis tractable, explanation functions can assume the following:

∀g̅, g̅′ ∈ [symbol] : Ψ(g̅)⊆g̅′⇒(g′)=Ψ(ĝ) which implies I(Y; G̅| [symbol] _{Ψ(G̅)})=I(Y; G̅|Ψ(G̅)). This condition can hold for the ground truth explanation in various datasets studied in the explainability literature.

An explanation assisted (EA) learning rule L_{EA}= [symbol] consists of a family of mappings L_{EA,t}: (Tₜ, Ψ|Tₜ(·))→ƒ(·), where Tₜ, t∈ [symbol] is the training set, Ψ(·) is an explanation function and Ψ|Tₜ(·) is the restriction to the training set, [symbol] is a set of integers.

At a high level, for a given task, P_{Y,G} an explanation function (explainer) Ψ(·) maps the input graph G to an explanation subgraph Gₑₓₚ. The subgraph is a good explanation if it is minimal and sufficient with respect to G. The minimality of the subgraph can be measured in terms of its number of edges (size). Ψ(G) is minimal if [symbol] (|Ψ(G)|) is as small as possible. Sufficiency means that the posterior distribution of the label Y does not change significantly if we are given that Ψ(G) is a subgraph of G instead of the complete realization of G. The explanation subgraph is sufficient if d_{TV}(P_{Y|G=g}, P_{Y|Ψ(g)⊆G}) is small for all g∈ [symbol] , where d_{TV} denotes the total variation distance. Consequently, for given parameters s∈ [symbol] and κ>0, the mapping Ψ(·) is an (s, κ)-explainer for the task P_{Y,G} if:

$${d}_{TV} ( {P}_{Y \vert G = g} , {P}_{Y \vert Ψ ( g ) ⊆ G} ) \leq κ , ∀ g ∈ G and E ( \vert Ψ ( G ) \vert ) \leq s . \qquad ( 1 )$$

If an (s, κ)-explainer exists, then the task is (s, κ)-explainable. Note that for any κ≥0 and any given classification task P_{Y,G}, the task is trivially ( [symbol] (|G|), κ)-explainable since the graph itself can be taken as its explanation, i.e., Ψ(G)=G. Furthermore, in most practical scenarios, input graphs contain redundant edges, and consequently, the tasks are (s, κ)-explainable for an s which is strictly smaller than [symbol] (|G|).

In block 120, the explainer subgraphs can be transformed into perturbed subgraphs by utilizing explanation assisted empirical risk minimization (EA-ERM) learned by the GNN to generate an augmented training dataset.

EA learning rules may significantly improve sample complexity if the Bayes error rate of the task is small enough. However, the learning rule should distinguish between the original training data and its EA perturbations to achieve the potential improvements. This observation aligns with recent observations in in the context of other transformation invariances such as rotations and scalings. This phenomenon is explained with the following example.

Consider the hypothesis class H which consists of all classifiers that classify their input only based on the number of edges in the graph. That is,

$$H = \{ f ( \cdot ) \vert ∀ G , {G}^{′} : \vert G \vert = \vert {G}^{′} \vert \to f ( G ) = f ( {G}^{′} ) \} .$$

Furthermore, consider the following binary classification problem. Let P_{Y}(0)=P_{Y}(1)=½, and let the graphs associated with label zero consist of the collection B₀={G||G|=n, β∃i∈[n]: Cᵢ⊆G and ∃j⊆[n]: Dⱼ⊆G}, where n>10 is fixed, Cᵢ, i∈[n] denotes a cycle of size i, and D; denotes a star of size j, where a star is a subgraph where all vertices are connected to a specific vertex called the center, and there are no edges between the rest of the vertices. Thus, B₀ consists of all graphs with exactly n edges and at least one cycle but no stars. Similarly, let the graphs associated with label one be given by B₁={G∥G|=n+1, ∃i∈ [n+1]: Cᵢ⊆G and ⊆j⊆[n+1]: Dⱼ⊆G}}. B₁ consists of all graphs with exactly n+1 edges that do not contain a cycle but contain a star. Let

$$α = \frac{1}{n} ,$$

and define

$$Ψ ( G ) {=}^{Δ} \{ \begin{matrix}{C}_{i} , if ∃ i : {C}_{i} ⊆ G \\ {D}_{i} , if ∃ i : {D}_{i} ⊆ G\end{matrix} ,$$

where κ=γ=0.

For DA-ERM to achieve zero error, it would have a sample complexity that can grow arbitrarily large as it needs to observes all possible explanation outputs. DA-ERM cannot distinguish between the augmented elements of B₀ and the original elements of B₁ and vice versa since they may have the same number of edges. Additionally, DA-ERM may achieve high error probability on the original data distribution if the elements of the augmented dataset are out-of-distribution with respect to P_{G}.

To overcome the issues with DA-ERM, the present embodiments generate explanation-preserving perturbations of the original input graph G, such that Gₑₓₚ⊆Gᵢ. G and Gᵢ are passed through the GNN f(·) to produce output labels Ŷ and Ŷₗ respectively. The graphs (G and Gᵢ) and the respective output labels Ŷ and Ŷₗ can be formed as the augmented training dataset. This method is effective in generating in-distribution graphs. This process is further explained in the following algorithm:

Explanation-preserving perturbation function Π(·):

Input: a graph G, explainer Ψ(·), hyper-parameter α1.
G^{c} = G − Ψ (G) # Compute the non-explanation subgraph
E_{α}_{₁} (G^{c}) = sample α₁ edges from G^{c}
Return E_{α}_{₁} (Gc) + Ψ(G)
end algorithm.

In block 121, edges from non-explanation subgraphs, computed using the explanation subgraphs, can be sampled to append to explanation subgraphs.

In an embodiment, the sampling algorithm can include random sampling, local sampling, depth-first sampling, breadth-first sampling. The hyper-parameter can include the neighborhood of nodes to be sampled.

In block 123, the augmented training set can be generated from perturbed subgraphs based of the union of the training set and the unions of a mapping of unlabeled graphs having associated labeled. To generate the augmented training dataset from perturbed subgraphs, the following can be performed. Consider a task P_{Y,G}, an explainer function and a parameter. An explanation-preserving perturbation S is a mapping: S^{α}(G) [symbol] {G′Ψ(G)⊆G′, |εΔε′|≤α|ε|}, where ε and ε′ are the edge sets of G and G′, respectively. The EA augmented training set can be defined as: T_{aug} [symbol] TU (U_{(G,Y)∈T}{(G′, Y)|G′∈S^{α}(G)}), where T is a training set, an explainer function Ψ(·), and hyperparameter α>0.

In an embodiment, the explainer function can be pre-determined by utilizing explainer models such as GNNExplainer, PGExplainer, etc. In another embodiment, a training dataset transformed with data augmentation techniques can be further transformed by using EA-ERM. For example, a training dataset is augmented with edge deletion. The resulting training dataset from edge deletion can be processed with EA-ERM to generate augmented training dataset.

The fundamental idea in data augmentation (DA) techniques is that in many application domains, there are label-preserving transformations that can be applied to enlarge the training set and facilitate generalization. For certain classes of graph learning tasks, graph transformations that only alter the nonexplanation subgraphs are label-preserving with high probability. To elaborate, assume a task with small Bayes error rate, so that the input graph G accurately captures the label Y. Then, if the task is explainable, from equation (1) it follows that for two input graphs G and G′, if the explanation Ψ(G) is a subgraph of G′, then G and G′ have the same label, with high probability. Thus, such label-preserving transformations can be used for EA-DA. The label-preserving property depends on the Bayes error rate, and if the error rate is high, then such transformations may not be label-preserving.

The relationship between the Bayes error rate, explainability, and perturbation invariance can be quantified. Let κ, γ>0 such that γ+2κ≤1 and let s∈N. Then, for any (κ, s)-explainable task PYG with Bayes error γ, the following holds:

Σ_{g}_{ₑₓₚ}P(Ψ(G)=gₑₓₚ)P(Y≠Y′|Ψ(G)=gₑₓₚ, gₑₓₚ⊆G′)≤−γ²−2κ²+2γ+3κ−3γκ, where Ψ(·) is a (κ, s)-explanation function for P_{Y,G}, Y and Y′ are the labels associated with G and G′, respectively, and (G, Y) and (G′, Y′) are generated independently and according to P_{Y,G}.

Additionally, the relationship between perturbation-invariance and explainability can be quantified. Let ϵ, δ, κ, γ ∈ (0, 1). The sample complexity of (ϵ, δ, κ, γ)-probably approximately correct (PAC) learning of H with respect to the explanation function Ψ(·), denoted by m_{EA}(ϵ, δ, κ, γ; H, Ψ), is defined as the smallest number of training samples m ∈ [symbol] for which there exists an explanation-assisted learning rule L such that, for every s∈ [symbol] and (s, κ)-explainable task P_{Y,G} with Bayes error rate less than or equal to γ:

$$P ( e r {r}_{{P}_{Y , G}} ( L ( T ) ) \leq {inf}_{f ∈ H} e r {r}_{{P}_{Y , G}} ( f ) + ϵ ) \geq 1 - δ ,$$

where err_{P}_{_{Y,G}}(f) is the statistical error of f(·) for the task P_{Y,G}, and |T|=m. If no such m exists, then the sample complexity is infinite.

The parameters (ϵ, δ) used in the standard PAC formulation, the explainability parameter κ, and the sample complexity can be parameterized by an upper-bound on the Bayes error rate γ. If γ=1, the agnostic PAC settings can be recovered; if γ=0, the task is deterministic, and if the optimal (zero-error) classifier is in the hypothesis class, the realizable PAC settings can be recovered. The explicit dependence on γ is needed to derive the bounds on EA-DA sample complexity in the sequel.

The sample complexity of EA-ERM can be expressed in terms of the explanation-assisted Vapnik-Chervonenkis (VC) dimension. Given an explanation function Ψ(·) and hypothesis class H, the explanation-assisted VC dimension VC_{EA}(H, Ψ) is defined as the largest integer k for which there exists a collection of graphs [symbol] ={g₁, g₂, . . . , gₖ} such that Ψ(gᵢ)≠Ψ(gⱼ) for all i≠j, and every labeling of [symbol] is realized by the hypothesis class H. I (G)=G can be the identity function. VC(H) [symbol] VC_{EA}(H, I) is the standard VC dimension, as it aligns with the notion of VC dimension considered in traditional PAC learnability analysis.

The following provides a simple example in which the standard VC dimension, VC(H), can be arbitrarily larger than the explanation-assisted VC dimension VC_{EA}(H, Ψ(·)). Let Cᵢ, i∈ [symbol] denote the single-cycle graph with i vertices, where the vertex set is V=[i] and the edge set is ε={(j,j+1), j∈[i−1]}U{(i, 1)}. A binary classification problem can be constructed as follows. Let the graphs associated with label zero belong to the collection B₀={CᵢUC₃, i>5} and those associated with label one belong to

$${B}_{1} = \{ {C}_{i} ⋃ {C}_{4} , i > 5 \} . Let Ψ ( G ) = \{ \begin{matrix}{C}_{3} , if G ∈ B 0 \\ {C}_{4} , otherwise\end{matrix} , κ = 0. Let {P}_{Y} ( 0 ) = {P}_{Y} ( 1 ) = 1 / 2 ,$$

and assume that for a given label Y=y, the graphs belonging to B_{y} are equally likely, i.e., P_{G|Y}(·|y) is uniform. Let H consist of all possible classifiers on the set B₀UB₁. VC(H)=∞. However, VC_{EA}(H, Ψ(·))=2 since there are only two explanation graphs, namely C₃ and C₄.

The sample complexity of explanation-assisted learning rules can be expressed in terms of VC_{EA}(H, Ψ(·)), as opposed to VC(H) which can be achieved by generic learning rules with the following. Let ϵ, δ, κ, γ∈(0, 1) such that

$$- {γ}^{2} - 2 {κ}^{2} + 2 γ + 3 κ - 3 γ κ \leq \frac{ϵ}{32} ,$$

then, for any hypothesis class H and explanation function Ψ(·), the following holds:

$${m}_{E A} ( ∈ , δ , κ , γ ; H , Ψ ) = O ( \frac{d}{{ϵ}^{2}} {log}^{2} d + \frac{1}{{ϵ}^{2}} ln \frac{1}{δ} ) where d {=}^{Δ} {VC}_{E A} ( H , Ψ ( \cdot ) ) .$$

ERM and EA-ERM both achieve zero error after observing at least one sample per label since all graphs of size n have label 0 and all graphs of size n+1 have label 1, and the hypothesis class decides based only on the number of edges. Additionally, ERM and EA-ERM have sample complexity equal to two.

In block 130, the GNN can be further trained with the augmented training dataset and the training dataset to perform downstream tasks using input data with corresponding labels.

To train the GNN with the augmented dataset, the following loss function can be employed. For a fixed parameter λ>0, the loss can be defined as:

$$Loss = CE ( Y , \hat{Y} ) + λ {\sum }_{i} CE ( Y , \hat{Y} ) \qquad ( 3 )$$

where CE is a reconstruction error such as cross entropy.

In block 131, a loss function can be learned based on a hyper parameter to limit the effect of the loss on the augmented dataset to the loss on data to be updated. To further alleviate the negative effects of out-of-distributed augmentations, we choose the hyperparameter λ (in Eq. (3)) small enough, so that the loss on the (potentially out-of-distribution) augmented data does not dominate the loss on the original data to be updated,

$$λ = 1 .$$

By training the GNN with the augmented training dataset, the data efficiency and accuracy of the GNNs training and the GNNs' learning of target datasets are increased when compared to other augmentation techniques (e.g., edge inserting, edge dropping, node dropping, feature dropping, mixup, etc.). In contrast, the performance of the GNN worsens when the GNN is trained with out of distribution augmentations which can result by performing other augmentation techniques through random edge addition, removal, etc.

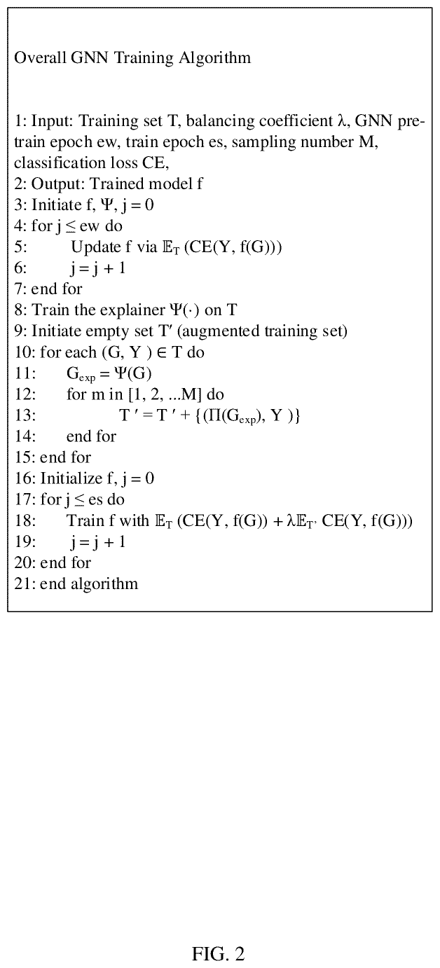

The overall training algorithm of the GNN is shown in more detail in FIG. 2.

Referring now to FIG. 2, a block diagram showing the overall algorithm for training the GNN for explanation-assisted data augmentation for graph neural network training, in accordance with an embodiment of the present invention.

Lines 4-7 includes the training algorithm for training the GNN to generate labeled graphs from training data. Lines 8-15 includes the training algorithm for training the GNN to generate perturbed subgraphs and the augmented training dataset. Lines 16-21 includes the training algorithm for training the GNN with the augmented training dataset.

Referring back now to FIG. 1. After training the GNN with the augmented training dataset, the GNN can be utilized to perform downstream tasks. The downstream tasks can include node classification, graph classification, graph visualization, link prediction.

Node classification can determine the labeling of samples (represented as nodes) by looking at the labels of their neighbors. To perform node classification, models can be trained in a semi-supervised way, with only a part of the graph being labeled. One example of downstream tasks for node classification is document classification. For example, a technical paper can cite other related papers. The cited papers can belong to similar research area. In this citation network, the citation information from each paper can be leveraged in addition to its own textual content. A dataset can be generated into a network of papers for predicting paper labels.

Graph classification can classify a whole graph into different categories. applications for graph classification can include determining whether a protein is an enzyme or not in bioinformatics, categorizing documents in natural language processing (NLP) in which a node is a word or a token of a document, or social network analysis.

Graph visualization can generate visual representation of graphs that reveals structures and anomalies that may be present in the data and helps the user to understand the graphs.

Link prediction can understand the relationship between entities in graphs to predict connections between entities such as inferring social interactions or suggesting possible connection to users in social networking applications. Link prediction can be utilized to predict criminal associations.

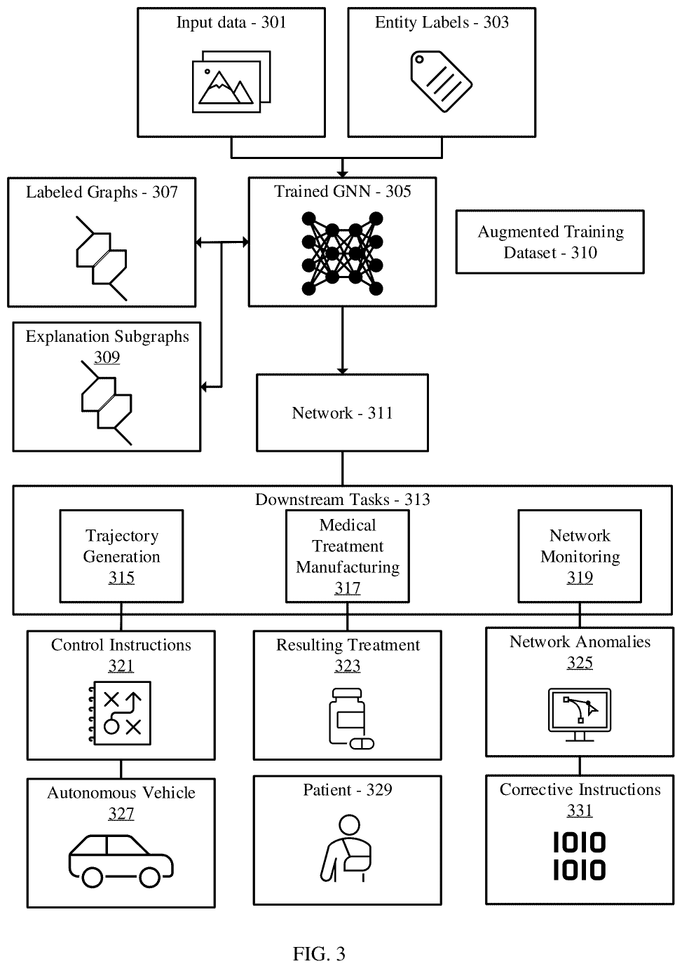

The downstream tasks is described in more detail in FIG. 3

Referring now to FIG. 3, a block diagram showing a system implementing practical applications for explanation-assisted data augmentation for graph neural network training, in accordance with an embodiment of the present invention.

In system 300, a trained GNN 305 can be utilized to process input data 301 and entity labels 303 to generate labeled graphs 307 and explanation subgraphs 309. The labeled graphs 307 and explanation subgraphs 309 can be processed by the trained GNN 305 to generate augmented training dataset 310 which can be used to further train the trained GNN 305 to perform downstream tasks 313. The trained GNN 305 can generate outputs for the downstream tasks which can be transmitted to respective end-user computing systems through a network 311. The downstream tasks 313 can include trajectory generation 315, medical treatment manufacturing 317 and network monitoring 319.

In an embodiment, for trajectory generation 315, the input data 301 and entity labels 303 can be obtained from a traffic scene using sensors within an autonomous vehicle 327. The labeled graphs 307 and explanation subgraphs 309 can be generated by the trained GNN 305 from the traffic scene. The labeled graphs 307 can include semantic information (e.g., distance, speed, previous driver behavior, previous travel histories, etc.) of the autonomous vehicle 327 in relation to the entities (e.g., road, intersection, vehicles, pedestrians, buildings, traffic signs, etc.). The explanation subgraphs 309 can include explanations on potential driving behaviors (e.g., speeding, slowing down, collisions, etc.) within the traffic scene.

In an embodiment, due to the training with the augmented training dataset 310, the trained GNN 305 can be implemented within a computer system within the autonomous vehicle 327 due to the decreased sample complexity of the augmented training dataset 310.

In another embodiment, the trained GNN 305 can generate control instructions 321 to control (e.g., slow down, speed up, change direction, etc.) the autonomous vehicle 327 based on the processed traffic scene. This is also shown in block 135 in FIG. 1. In another embodiment, the trained GNN 305 can generate simulations of future traffic behavior based on past driver behavior and past processing of traffic scenes.

In another embodiment, for medical treatment manufacturing 317, input data 301 and entity labels 303 of chemical compositions of potential treatment compounds can be processed by the trained GNN 305 to generate labeled graphs 307 and explanation subgraphs 309. The explanation subgraphs 309 can represent why the potential treatment compounds can be used to treat a particular disease (e.g., decreases blood sugar concentration for diabetes, decreases blood pressure for hypertension, etc.). This is also shown in block 133 in FIG. 1.

In an embodiment, the trained GNN 305 can be used to provide the potential treatment compound to a treatment manufacturer to manufacture resulting treatment 323. To confirm the existence of the particular disease, explanation subgraphs 309 that represents target proteins identified to be related to the particular disease can be generated by the trained GNN 305 based on labeled graphs 307 that represents protein composition (e.g., deoxyribonucleic acid [DNA], etc.) of the patients generated by the trained GNN 305.

After confirming the existence of the particular disease (e.g., diabetes, hypertension, etc.) and after confirming that the resulting treatment 323 is effective to treat the particular disease, the resulting treatment 323 can be administered to the patient 329.

In another embodiment, for network monitoring 319, the trained GNN 305 can monitor network data streams and can process input data 301 (e.g., network data logs, network data streams as time-series data, etc.) and corresponding entity labels 303 (e.g., labels for network hardware, internet protocol (IP) addresses, user data, entity data, etc.) to generate labeled graphs 307 and explanation subgraphs 309. The labeled graphs 307 can represent the network connections and the semantic information (e.g., status, bandwidth, service level status, etc.) of the entities. The explanation subgraphs 309 can represent the target behavior being monitored such as decreased bandwidth, increased hit rate from a particular IP address, etc. In another embodiment, the explanation subgraphs 309 can represent network anomalies 325. Based on the explanation subgraphs 309 and the labeled graph 307, the trained GNN 305 can generate corrective instructions 331 to correct the issues caused by the network anomalies 325 such as blocking the IP address of the entity causing the increased hit rate, increasing the bandwidth of the observed entity, etc.

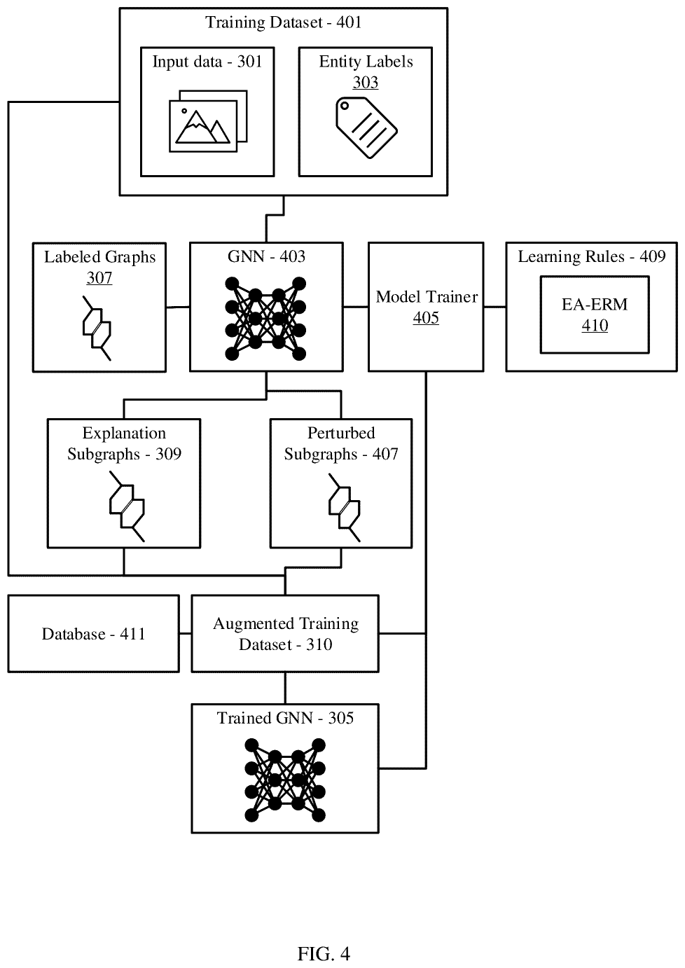

Referring now to FIG. 4, a block diagram showing software and hardware components of a computer system for explanation-assisted data augmentation for graph neural network training, in accordance with an embodiment of the present invention.

In an embodiment, training dataset 401 can include input data 301 and entity labels 303. The GNN 403 can be trained using learning rules 409 to generate labeled graphs 307 from the training dataset 401. The GNN 403 can be trained using the learning rules 409 to generate explanation subgraphs 309. In another embodiment, the GNN 403 can utilize explainer functions such as GNNExplainer, PGExplainer, etc., to generate the explanation subgraphs 309. In an embodiment, the GNN 403 can be trained using the EA-ERM 410 to generate perturbed subgraphs 407. The explanation subgraphs 309, perturbed subgraphs 407 and the training dataset can be processed to generate augmented training dataset 310. The augmented training dataset 310 can be saved to a database 411. The database 411 can save previous iterations of training including model parameters updated during the training process, training dataset used, performance measurements of the GNN 403 and the trained GNN 305. The augmented training dataset 310 can be used by the model trainer 405 train the GNN 403 to obtain a trained GNN 305.

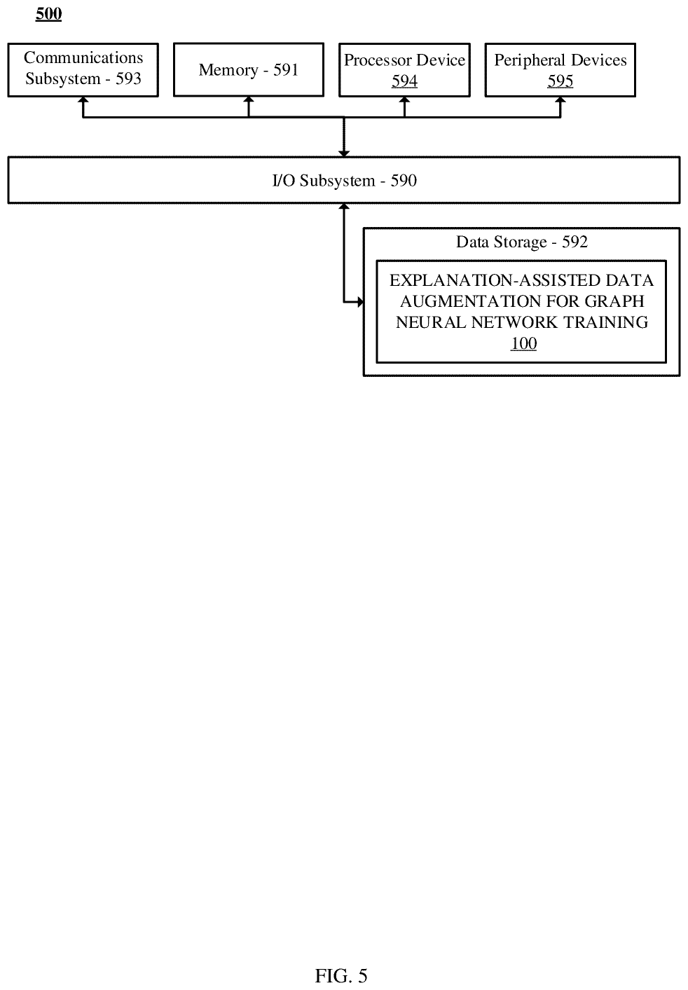

Referring now to FIG. 5, a block diagram showing a computer system for explanation-assisted data augmentation for graph neural network training, in accordance with an embodiment of the present invention.

The computing device 500 illustratively includes the processor device 594, an input/output (I/O) subsystem 590, a memory 591, a data storage device 592, and a communication subsystem 593, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 591, or portions thereof, may be incorporated in the processor device 594 in some embodiments.

The processor device 594 may be embodied as any type of processor capable of performing the functions described herein. The processor device 594 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 591 may be embodied as any type of volatile or non-volatile memory or data storage capable of performing the functions described herein. In operation, the memory 591 may store various data and software employed during operation of the computing device 500, such as operating systems, applications, programs, libraries, and drivers. The memory 591 is communicatively coupled to the processor device 594 via the I/O subsystem 590, which may be embodied as circuitry and/or components to facilitate input/output operations with the processor device 594, the memory 591, and other components of the computing device 500. For example, the I/O subsystem 590 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 590 may form a portion of a system-on-a-chip (SOC) and be incorporated, along with the processor device 594, the memory 591, and other components of the computing device 500, on a single integrated circuit chip.

The data storage device 592 may be embodied as any type of device or devices configured for short-term or long-term storage of data such as, for example, memory devices and circuits, memory cards, hard disk drives, solid state drives, or other data storage devices. The data storage device 592 can store program code for explanation-assisted data augmentation for graph neural network training 100. Any or all of these program code blocks may be included in a given computing system.

The communication subsystem 593 of the computing device 500 may be embodied as any network interface controller or other communication circuit, device, or collection thereof, capable of enabling communications between the computing device 500 and other remote devices over a network. The communication subsystem 593 may be configured to employ any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 595. The peripheral devices 595 may include any number of additional input/output devices, interface devices, and/or other peripheral devices. For example, in some embodiments, the peripheral devices 595 may include a display, touch screen, graphics circuitry, keyboard, mouse, speaker system, microphone, network interface, and/or other input/output devices, interface devices, GPS, camera, and/or other peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be employed. Moreover, additional processors, controllers, memories, and so forth, in various configurations can also be utilized. These and other variations of the computing device 500 are readily contemplated by one of ordinary skill in the art given the teachings of the present invention provided herein.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

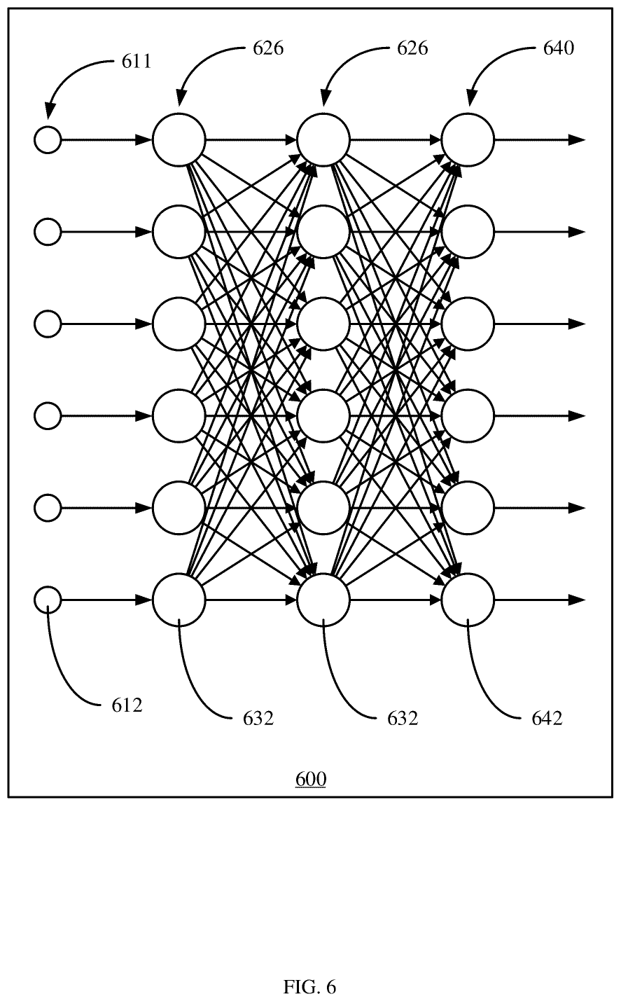

Referring now to FIG. 6, a block diagram showing a structure of deep neural networks for explanation-assisted data augmentation for graph neural network training, in accordance with an embodiment of the present invention.

A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the inputted data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types and may include multiple distinct values. The network can have one input neurons for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

The deep neural network 600, such as a multilayer perceptron, can have an input layer 611 of source neurons 612, one or more computation layer(s) 626 having one or more computation neurons 632, and an output layer 640, where there is a single output neuron 642 for each possible category into which the input example could be classified. An input layer 611 can have a number of source neurons 612 equal to the number of data values 612 in the input data 611. The computation neurons 632 in the computation layer(s) 626 can also be referred to as hidden layers, because they are between the source neurons 612 and output neuron(s) 642 and are not directly observed. Each neuron 632, 642 in a computation layer generates a linear combination of weighted values from the values output from the neurons in a previous layer, and applies a non-linear activation function that is differentiable over the range of the linear combination. The weights applied to the value from each previous neuron can be denoted, for example, by w₁, w₂, . . . wₙ₋₁, wₙ. The output layer provides the overall response of the network to the inputted data. A deep neural network can be fully connected, where each neuron in a computational layer is connected to all other neurons in the previous layer, or may have other configurations of connections between layers. If links between neurons are missing, the network is referred to as partially connected.

Training a deep neural network can involve two phases, a forward phase where the weights of each neuron are fixed and the input propagates through the network, and a backwards phase where an error value is propagated backwards through the network and weight values are updated. The computation neurons 632 in the one or more computation (hidden) layer(s) 626 perform a nonlinear transformation on the input data 612 that generates a feature space. The classes or categories may be more easily separated in the feature space than in the original data space.

The neural network 600 can be trained to learn the EA-ERM learning rule. In an embodiment, the computation layers 626 of the neural network (e.g., a graph neural network 403) can learn relationships of the semantic data represented in the nodes of a label graph 307 and learn perturbations for the semantic data. The output layer 640 of the neural network 600 can then generate perturbed subgraphs 407 of the labeled graph 307 based on the learned relationships and perturbations.

In another embodiment, based on the perturbed subgraphs and pre-learned generative text processing capabilities, the neural network 600 can generate an augmented training dataset 310. The computation layers 626 can learn the correlation or gaps between the previous training dataset 401 and the semantic information within the perturbed subgraphs 407. The output layer 640 of the neural network 600 can output generated text that fills in the gaps and generate the augmented training dataset 310.

In another embodiment, because the computation layers 626 of the neural network 600 learned the relationships of the semantic data, the computation layers 626 of the neural network 600 can also learn explanations within the labeled graph 307. The output layer 640 of the neural network 600 can then generate explanation subgraphs 309, depending on the context, of a labeled graph 307 which can be used to perform downstream tasks 313.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for training a graph neural network (GNN), comprising:
training the GNN using a training dataset to generate explainer subgraphs of labeled graphs;
transforming the explainer subgraphs into perturbed subgraphs by utilizing explanation assisted empirical risk minimization (EA-ERM) learned by the GNN to generate an augmented training dataset; and
training the GNN with the augmented training dataset and the training dataset to perform downstream tasks using input data with corresponding labels.

2. The computer-implemented method of claim 1, wherein the downstream tasks further comprise generating medical treatments based on the explanation graphs of chemical compounds generated by the GNN from images and labels.

3. The computer-implemented method of claim 1, wherein the downstream tasks further comprise generating a trajectory to control an autonomous vehicle based on previous travel histories and images of traffic scenes learned by the GNN.

4. The computer-implemented method of claim 1, wherein training the GNN using the training dataset further comprises generating explainer subgraphs by employing an explanation assisted learning rule learned by the GNN through training.

5. The computer-implemented method of claim 1, wherein training the GNN using the training dataset further comprises learning a graph classification loss function to generate labeled graphs.

6. The computer-implemented method of claim 1, wherein transforming the explainer subgraphs further comprises sampling edges from non-explanation subgraphs to append to explanation subgraphs.

7. The computer-implemented method of claim 1, wherein transforming the explainer subgraphs further comprises, generating the augmented training set from perturbed subgraphs based on a union of the training set and the unions of a mapping of unlabeled graph having associated labels.

8. The computer-implemented method of claim 1, wherein training the GNN with the augmented training dataset further comprises learning a loss function based on a hyperparameter to limit the loss on the augmented dataset to the loss on data to be updated.

9. A system for training a graph neural network (GNN), comprising:
a memory device;
one or more processor devices operatively coupled with the memory device to perform operations including:
training the graph neural network (GNN) using a training dataset to generate explainer subgraphs of labeled graphs;
transforming the explainer subgraphs into perturbed subgraphs by utilizing explanation assisted empirical risk minimization (EA-ERM) learned by the GNN to generate an augmented training dataset; and
training the GNN with the augmented training dataset and the training dataset to perform downstream tasks using input data with corresponding labels.

10. The system of claim 9, wherein the downstream tasks further comprise generating medical treatments based on the explanation graphs of chemical compounds generated by the GNN from images and labels.

11. The system of claim 9, wherein the downstream tasks further comprise generating a trajectory to control an autonomous vehicle based on previous travel histories and images of traffic scenes learned by the GNN.

12. The system of claim 9, wherein training the GNN using the training dataset further comprises generating explainer subgraphs by employing an explanation assisted learning rule learned by the GNN through training.

13. The system of claim 9, wherein training the GNN using the training dataset further comprises learning a graph classification loss function to generate labeled graphs.

14. The system of claim 9, wherein transforming the explainer subgraphs further comprises sampling edges from non-explanation subgraphs to append to explanation subgraphs.

15. The system of claim 9, wherein transforming the explainer subgraphs further comprises generating the augmented training set from perturbed subgraphs based on a union of the training set and the unions of a mapping of unlabeled graph having associated labels.

16. The system of claim 9, wherein training the GNN with the augmented training dataset further comprises learning a loss function based on a hyperparameter to limit the loss on the augmented dataset to the loss on data to be updated.

17. A non-transitory computer program product comprising a computer-readable storage medium including a program code for training a graphical neural network (GNN), wherein the program code when executed on a computer causes the computer to perform operations including:
training the GNN using a training dataset to generate explainer subgraphs of labeled graphs;
transforming the explainer subgraphs into perturbed subgraphs by utilizing explanation assisted empirical risk minimization (EA-ERM) learned by the GNN to generate an augmented training dataset; and
training the GNN with the augmented training dataset and the training dataset to perform downstream tasks using input data with corresponding labels.

18. The non-transitory computer program product of claim 17, wherein the downstream tasks further comprise generating medical treatments based on the explanation graphs of chemical compounds generated by the GNN from images and labels.

19. The non-transitory computer program product of claim 17, wherein the downstream tasks further comprise generating a trajectory to control an autonomous vehicle based on previous travel histories and images of traffic scenes learned by the GNN.

20. The non-transitory computer program product of claim 17, wherein training the GNN using the training dataset further comprises generating explainer subgraphs by employing an explanation assisted learning rule learned by the GNN through training.
